SEQUENCE LISTING (13 sequences)

SEQ ID NO: 1 - DNA, 24 bp, Artificial Sequence
  sense forward primer
tatgggttggttagttttgttttg

SEQ ID NO: 2 - DNA, 21 bp, Artificial Sequence
  sense reverse primer
cacattcaccttaccccacaa

SEQ ID NO: 3 - DNA, 24 bp, Artificial Sequence
  antisense forward parimer
gtttggattaggagtttattgata

SEQ ID NO: 4 - DNA, 19 bp, Artificial Sequence
  antisense reverse primer
ttccccacactatctcaat

SEQ ID NO: 5 - DNA, 19 bp, Artificial Sequence
  prmt5 siRNA
ggacctgagagatgatata

SEQ ID NO: 6 - DNA, 19 bp, Artificial Sequence
  prmt5 siRNA
gaggattgcagtggctctt

SEQ ID NO: 7 - DNA, 18 bp, Artificial Sequence
  prmt5 siRNA scrambled
acgtctactatcgacccc

SEQ ID NO: 8 - DNA, 20 bp, Artificial Sequence
  hprt sense primer
atggacaggactgaacgtct

SEQ ID NO: 9 - DNA, 20 bp, Artificial Sequence
  hprt antisense primer
cttgcgaccttgaccatctt

SEQ ID NO: 10 - DNA, 21 bp, Artificial Sequence
  gamma globin sense primer
agctttggcaacctgtcctct

SEQ ID NO: 11 - DNA, 21 bp, Artificial Sequence
  gamma globin antisense primer
ggccactccagtcaccatctt

SEQ ID NO: 12 - DNA, 1914 bp, Homo sapiens
atggcggcgatggcggtcgggggtgctggtgggagccgcgtgtccagcgggagggacctgaattgcgtccccgaaatagctgacacactaggggctgtggccaagcaggggtttgatttcctctgcatgcctgtcttccatccgcgtttcaagagggagttcattcaggaacctgctaagaatcggcccggtccccagacacgatcagacctactgctgtcaggaagggactggaatacgctaattgtgggaaagctttctccatggattcgtccagactcaaaagtggagaagattcgcaggaactccgaggcggccatgttacaggagctgaattttggtgcatatttgggtcttccagctttcctgctgccccttaatcaggaagataacaccaacctggccagagttttgaccaaccacatccacactggccatcactcttccatgttctggatgcgggtacccttggtggcaccagaggacctgagagatgatataattgagaatgcaccaactacacacacagaggagtacagtggggaggagaaaacgtggatgtggtggcacaacttccggactttgtgtgactatagtaagaggattgcagtggctcttgaaattggggctgacctcccatctaatcatgtcattgatcgctggcttggggagcccatcaaagcagccattctccccactagcattttcctgaccaataagaagggatttcctgttctttctaagatgcaccagaggctcatcttccggctcctcaagttggaggtgcagttcatcatcacaggcaccaaccaccactcagagaaggagttctgctcctacctccaatacctggaatacttaagccagaaccgtcctccacctaatgcctatgaactctttgccaagggctatgaagactatctgcagtccccgcttcagccactgatggacaatctggaatctcagacatatgaagtgtttgaaaaggaccccatcaaatactctcagtaccagcaggccatctataaatgtctgctagaccgagtaccagaagaggagaaggataccaatgtccaggtactgatggtgctgggagcaggacggggacccctggtgaacgcttccctgcgggcagccaagcaggccgaccggcggataaagctgtatgctgtggagaaaaacccaaatgccgtggtgacgctagagaactggcagtttgaagaatggggaagccaagtgaccgtagtctcatcagacatgagggaatgggtggctccagagaaagcagacatcattgtcagtgagcttctgggctcatttgctgacaatgaattgtcgcctgagtgcctggatggagcccagcacttcctaaaagatgatggtgtgagcatccccggggagtacacttcctttctggctcccatctcttcctccaagctgtacaatgaggtccgagcctgtagggagaaggaccgtgaccctgaggcccagtttgagatgccttatgtggtacggctgcacaacttccaccagctctctgcaccccagccctgtttcaccttcagccatcccaacagagatcctatgattgacaacaaccgctattgcaccttggaatttcctgtggaggtgaacacagtactacatggctttgccggctactttgagactgtgctttatcaggacatcactctgagtatccgtccagagactcactctcctgggatgttctcatggtttcccatcctcttccctattaagcagcccataacggtacgtgaaggccaaaccatctgtgtgcgtttctggcgatgcagcaattccaagaaggtgtggtatgagtgggctgtgacagcaccagtctgttctgctattcataaccccacaggccgctcatataccattggcctctag

SEQ ID NO: 13 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

(19) United States
(12) Patent Application Publication
(10) Pub. No.: US 2010/0061989 A1
(43) Pub. Date: Mar. 11, 2010

(54) METHOD OF TREATMENT AND PROPHYLAXIS

(75) Inventors: Stephen M. Jane, Victoria (AU); Quan Zhao, Victoria (AU); Gerhard Rank, Victoria (AU)

Correspondence Address: KNOBBE MARTENS OLSON & BEAR LLP, 2040 MAIN STREET, FOURTEENTH FLOOR, IRVINE, CA, 92614, US

(73) Assignee: MELBOURNE HEALTH, PARKVILLE, VICTORIA (AU)

(21) Appl. No.: 12/515,942

(22) PCT Filed: Nov. 21, 2007
(86) PCT No.: PCT/AU2007/001788

§ 371 Date: Oct. 30, 2009

Related U.S. Application Data
(60) Provisional application No. 60/860,693, filed on Nov. 21, 2006.

(51) Int. Cl. A61K 39/395; A61K 31/7052; A61P 7/04; A61K 38/02; A61K 38/45

(52) U.S. Cl. 424/135.1; 514/44 A; 424/158.1; 514/2; 424/94.5; 424/146.1

(57) ABSTRACT

The present invention relates generally to a method for the treatment of a hemoglobinopathic condition in mammalian subjects such as humans and medicaments useful for same.

### FIELD

The present invention relates generally to a method for the treatment of a hemoglobinopathic condition in mammalian subjects such as humans and medicaments useful for same.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Hemoglobin is a major protein in red blood cells and is essential for the transport of oxygen from the lungs to the tissues. Defects involving the human globin genes are the most prominent genetic disorders worldwide affecting about 10% of the global population.

These defects are collectively referred to as hemoglobinopathies or hemoglobinopathic conditions. Children afflicted with two of these disorcers, sickle cell disease (SCD) and β-thalassemia have significant morbidity and a markedly reduced life expectancy, particularly in under-developed countries. In SCD, a point mutation in the coding sequence in the gene encoding β-globin leads to the production of a protein with altered polymerisation properties, resulting in reduced deformability of the red cells resulting in blockage of microcapillaries. The clinical consequences of this include severe pain, bone death, stroke, renal failure, cognitive impairment and sudden death. In β-thalassemia, the adult globin chains are not produced, and children are transfusion dependent for life (where available) with the resultant problems of tissue iron overload and the constant risk of septicaemia and transmission of blood borne disorders.

Gene silencing is a key feature in developmental regulation and in emergence of disease phenotypes. In fact, DNA methylation and repressive histone modifications play essential and often co-ordinated roles in gene silencing. Notwithstanding, direct links between these epigenetic alterations have been difficult to establish.

Epigenetic “conversation” between histones and DNA involving tyrosine methylation, histone deacetylation, and di- or tri-methylation of histone H3 at lysine 9 (H3K9me2, H3K9me3, respectively) has been implicated in gene silencing (Fuks, Curr. Opin. Genet. Dev 15:490, 2005). In some settings, DNA methylation has been shown to influence the histone modification pattern, with DNA methyltransferases and methyl-CpG-binding domain proteins involved in recruitment of repressor complexes containing histone deacetylases (Bird, Genes Dev 16:6, 2002). Conversely, studies in fungi, plants and mammals have suggested that methylation of H3K9 is a prerequisite for subsequent DNA methylation (Tamaru et al, Nat. Genet. 34:75, 2003; Jackson et al, Nature 416:556, 2002; Lehnertz et al, Curr. Biol. 13:1192, 2003), and the functional link between these processes appears to be due to a physical association between the histone methylation system and DNA methyltransferases (Lehnertz et al, supra 2003). Similar links between methylation of histone 3 at lysine 27 (H3K27) and DNA methylation have recently been proposed (Fuks, supra 2005).

The β-globin locus has served as a paradigm for analysing the role of epigenetic modifications in the regulation of tissue and developmentally-specific gene expression (Litt et al, Science 293:2453, 2001; Schneider et al, Nat. Cell. Biol. 6:73, 2004; Bulger et al, Mol Cell Biol 23:5234, 2003; Johnson et al, Mol. Cell. 8:465, 2001). In both humans and primates, the fetal (γ)-globin genes are progressively silenced after birth, displaying methylation of a cluster of CpG dinucleotides in the proximal promoters and 5′ untranslated regions in adult bone marrow (van der Ploeg and Flavell, Cell 19:947, 1980). Reversal of this methylation is associated with fetal globin gene reactivation (Lavelle et al, Exp. Hematol 34:339, 2006).

In accordance with the present invention, the components involved in regulation of expression of fetal globin genes are elucidated enabling rationale drug design to induce expression of silenced fetal globin genes in the treatment of hemoglobinopathies.

### SUMMARY

The present invention is predicated in part on the determination of the underlying mechanisms controlling the silencing of fetal gene expression after birth. In particular, the protein (PR) methyltransferase (MT), PRMT-5, is identified as the enzyme responsible for symmetric di-methylation of arginine 3 (R3) on histone H4 (H4R3me2s) and hence is a prerequisite for repressive histone modifications and DNA methylation. In accordance with the present invention, a PRMT-5-dependent complex comprising Dnmt3a, casein kinase IIα, Suv4-2oh1/2 and components of the MBD2/NuRD complex induces phosphorylation of H4S1, tri-methylation of H4K20, H3K9 and H3K27 and CpG methylation. This co-ordinated repression is dependent on the methyltransferase activity of PRMT-5, establishing a control role for this factor and the complex in mammalian gene silencing.

Accordingly, PRMT-5 or a co-factor associated therewith is a target for agents which antagonize levels or activity of PRMT-5 and/or its ability to participate in the PRMT-5-dependent complex. The PRMT-5-containing complex is also a target for antagonists as are other components in the complex. Antagonism of PRMT-5 or the PRMT-5-containing complex or of components therein enables repressed fetal γ-globin genes to be expressed providing means for treating hemoglobinopathies in mammalian subjects in particular humans, by the production of fetal γ-globin.

Hence, one aspect of the present invention contemplates a method for the treatment of a hemoglobinopathy in a mammalian subject, said method comprising administering to the subject an agent which disrupts or down-regulates the activity of a component of a PRMT-5-dependent, transcription-regulating complex or a gene encoding PRMT-5 or the other component the agent being administered for a time and under conditions sufficient for a suppressed fetal γ-globin gene to be expressed.

In a particular embodiment, the present invention provides a method for the treatment of a hemoglobinopathy in a mammalian subject, the method comprising administering to the subject an agent which disrupts or down-regulates the level or activity of PRMT-5 or other component in the PRMT-5-dependent, transcription-regulating complex said agent being administered for a time and under conditions sufficient for a suppressed fetal γ-globin gene to be expressed.

The present invention further relates to a method for reactivating expression of a silenced γ-globin gene in a cell the method comprising contacting the cell with an agent which disrupts or down-regulates the activity of a component of a PRMT-5-dependent, transcription complex or a gene encoding PRMT-5.

The present invention is also directed to antagonists of a PRMT-5-dependent, transcription-regulating complex of a fetal γ-globin gene. In particular, the present invention provides an antagonist of PRMT-5 or gene encoding same or a component of the PRMT-5 complex or gene encoding such as a compound.

Such antagonists are proposed to be used in the manufacture of a medicament for the treatment of a hemoglobinopathy in a subject.

Particular subjects are humans.

The antagonists may be of the PRMT-5 protein or a co-factor thereof, or of the activity of the PRMT-5-containing complex or a component thereof or of gene expression of the gene encoding PRMT-5 or other component.

Reference to “activity” includes enzymatic activity and function of PRMT-5 or other component in the PRMT complex.

Pharmaceutical compositions, therapeutic protocols, research reagents and the like also form part of the present invention.

A summary of sequence identifiers used throughout the subject specification is provided in Table 1. The nucleotide sequence and corresponding amino acid sequence of human PRMT-5 are represented in SEQ ID NOs:12 and 13, respectively.

A histone target of methylation is defined by histone number followed by the amino acid residue which is methylated. Hence, HaXb is used to denote histone “a” is methylated at amino acid residue X “b”. A symmetric di- or tri-methylation is designated as Me2s or Me3s after the HaXb. Assymetric dimethylation is designated “Me2a”.

TABLE 1
Summary of sequence identifiers

| SEQUENCE ID NO: | DESCRIPTION |
|---|---|
| 1 | Human γ-globin Sense Forward Primer |
| 2 | Human γ-globin Sense Reverse Primer |
| 3 | Human γ-globin Antisense Forward Primer |
| 4 | Human γ-globin Antisense Reverse Primer |
| 5 | Synthetic PRMT-5 siRNA |
| 6 | Synthetic PRMT-5 siRNA |
| 7 | Synthetic PRMT-5 siRNA scrambled control |
| 8 | Human HPRT sense |
| 9 | Synthetic HRPT antisense |
| 10 | Human γ-globin sense |
| 11 | Synthetic γ-globin antisense |
| 12 | Nucleotide sequence encoding human PRMT-5 |
| 13 | Amino acid sequence encoding human PRMT-5 |

### BRIEF DESCRIPTION OF THE FIGURES

FIGS. 1A through E are photographic representations showing that PRMT-5 and NF-E4 interact and induce H4R3me2s at the γ-promoters. (A) PRMT-5 (α-PRMT-5) or pre-immune (PI) immunoprecipitates from cell extract from untransfected K562 cells were analyzed by western blotting using NF-E4 or PRMT-5 antibodies. (B) ³⁵S-labeled PRMT-5 was incubated with purified GST, and GST fusion proteins containing amino acids 1-48, 49-100, 101-179, and 1-179 (full length) of NF-E4 (bottom panel, Coomassie stain, marked with *) pre-adsorbed to glutathione-Sepharose beads. Eluted protein was visualized by autoradiography (top panel) after SDS-PAGE. Input represents 5% of the labeled PRMT-5 used in the assay. (C) Chromatin fractions from K562 cells were immunoprecipitated with either NF-E4 or PRMT-5 antibodies. No antibody and pre-immune sera served as the controls. The precipitated DNA was amplified with primers specific for the γ-promoters, or the control MyoD promoter. (D) FLAG-tagged wild type PRMT-5 (PRMT-5-f), and a methyltransferase-dead mutant (PRMT-5Δ-f) were expressed in K562 cells. FLAG immunoprecipitates from these cells and an untransfected control line were used for HMTase assays against purified histones (left panels). HA immunoprecipitates from K562 cells transfected with either PRMT-5-f or PRMT-5Δ-f and HA-NF-E4 were also assayed (right panel). Autoradiograph (upper panel), and Coomassie stained gel (lower panel) are shown for each. (E) Chromatin fractions from K562 cells expressing PRMT-5-f or PRMT-5-f were immunoprecipitated with pre-immune sera (PI), or a pan H4 antibody (H4 pan) followed by ab5823, which recognizes H4R3me2s and analyzed as in (C).

FIGS. 2 A through E are photographic and graphic representations showing that perturbed expression of PRMT-5 alters γ-globin gene expression and induces specific histone modifications at the γ-promoters. (A) Extracts from PRMT-5-f, PRMT-5Δ-f, or vector control K562 cells were analyzed by western blot with anti-FLAG antibody (bottom panel). RNA from these cells was analyzed by Northern blot with probes specific for the γ-globin genes and the control housekeeping gene, GAPDH. (B) K562 cells transfected with an expression vector containing either short interfering RNAs (siRNAs) (PRMT-5-kd), or a scrambled sequence (scr) were analyzed by western blot (with anti-PRMT-5, tubulin, and GATA-1 antibodies), and Northern analysis as in (A). (C) Chromatin fractions from PRMT-5-f and PRMT-5-kd K562 cells were immunoprecipitated with a range of antibodies to modified histone H4 or H3, and RNA polII. The precipitated DNA was subjected to quantitative PCR with primers specific for the γ-promoters, or the control MyoD or GATA-1 promoters. Quantitation of the relative levels of each modification is demonstrated in the bar graph in which the higher value was set at 1 for each pair. (D) and (E) ChIP was performed as in (C) with the indicated antibodies on chromatin derived from PRMT-5-f and PRMT-5Δ-f expressing K562 cells.

FIGS. 3 A through D are photographic, graphical and diagrammatical representations of the assembly of a PRMT-5-dependent repressor complex on the human γ-promoters that induces DNA methylation. (A) FLAG immunoprecipitates from PRMT-5-f K562 cells were analyzed by western blot with a range of antibodies to candidate protein partners. Immunoprecipitates with pre-immune sera served as the control. (B) Localization of complex components to the γ-promoters by ChIP. Chromatin fractions from PRMT-5-f K562 cells were immunoprecipitated with a range of antibodies to complex components identified in (A). The precipitated DNA was amplified with primers specific for the γ-promoters. (C) ChIP was performed as in (B) with the stated antibodies on chromatin derived from PRMT-5Δ-f expressing K562 cells. (D) Effect of perturbed PRMT-5 expression on DNA methylation at the human γ-genes. Each column shows the methylation status of individual CpG dinucleotides derived from sequence analysis of individual cloned PCR products of the γ-genes following bisulfite modification from PRMT-5-f, PRMT-5Δ-f, PRMT-5-kd, and the scrambled control (scr) K562 cells. The numbers at the top of the figure indicate the nucleotide positions of CpGs relative to the transcriptional start sites of the γ-globin genes.

FIGS. 4 A through C are graphical and photographic representations showing that PRMT-5 induced epigenetic modification of the γ-globin genes is developmentally-specific. (A) Real time RT-PCR of γ-globin gene expression in primary human erythroid progenitors from CB and adult BM standardized against HPRT. (B) Chromatin fractions from erythroid progenitors from CB and adult BM were immunoprecipitated with a pan H4 antibody followed by ab5823, which recognizes H4R3me2s, or RNA PolII. The precipitated DNA was amplified with primers specific for the γ-promoters. (C) Cellular localization of PRMT-5 in erythroid progenitors from CB and adult BM shown by immunofluorescence with anti-PRMT-5 antibody and DAPI nuclear counterstaining.

### DETAILED DESCRIPTION

All scientific citations, patents, patent applications and manufacturer's technical specifications referred to hereinafter are incorporated herein by reference in their entirety.

The present invention arose in the context of studying how fetal globin genes are regulated. The expression of such genes is subjected to gene silencing after birth mediated by repressive histone modifications and DNA methyltransferase, PRMT-5, mediates symmetric di-methylation of arginine 3(R3) on histone H4 (H4R3me2s) and inhibition of expression of fetal γ-globin gene expression (γ-genes). Hence, PRMT-5 plays an essential role in initiating co-ordinated repressive epigenetic events that culminate in DNA methylation and transcriptional silencing of the γ-genes. Assembly of a PRMT-5-dependent complex containing Dnmt3a, casein kinase IIa, Suv4-20h1/2, and components of the MBD2/NuRD complex induces the repressive markers phosphorylation of H4S1, tri-methylation of H4K20, K3K9 and K3K27, and CpG methylation. This co-ordinated repression is dependent on both the binding of PRMT-5 to the proximal γ-promoter, and its methylransferase activity.

Accordingly, it is proposed that the directed re-activation of human fetal γ-globin gene expression in subjects having, a hemoglobinopathic disorder will ameliorate the clinical severity of these disorders. Such directed re-activation is by targeting PRMT-5 activity or function, its ability to interact within the complex, the level of gene expression of the PRMT-5 gene or the level, activity or interactivity of any other componenet in the PRMT-5 complex such as Dnmt3 am caseine kinase IId, Suv4-2oh1/2 and components of the MBD2/NuRD complex.

It is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

As used in the subject specification, the singular forms “a”, “an” and “the” include plural aspects unless the context clearly dictates otherwise. Thus, for example, reference to “a disease condition” includes a single disease condition as well as two or more disease conditions; reference to “an active agent” includes a single active agent, as well as two or more active agents; reference to “the hemoglobinopathic condition” includes a single condition or multiple conditions; and so forth.

The terms “compound”, “agent”, “pharmacologically active agent”, “medicament”, “active” and “drug” are used interchangeably herein to refer to a chemical compound including a genetic molecule that induces a desired pharmacological and/or physiological effect. This effect includes the disruption of the repressive action of a complex comprising PRMT-5 on γ-globin gene expression or inhibition of the activity of PRMT-5 or a co-factor thereof, or down-regulating expression of a gene encoding PRMT-5 or other component of the PRMT-5 complex, modulating expression levels of a γ-globin gene as well as ameliorating the severity of symptoms of a hemoglobinopathy. All such terms preferably define antagonists. In addition, reference to “PRMT-5” includes PRMT-5 itself and any co-factors.

The present invention also extends to agonists of PRMT-5 or a complex comprising same. Such agonists are useful as research tools.

The term “agonist” as used herein refers to a molecule which promotes activity or levels of PRMT-5 or a complex comprising same and hence leading to repression of γ-gene expression. An “antagonist” inhibits PRMT-5 enzymatic activity or function or interactability with other components of the PRMT-5 complex.

A “hemoglobinopathy” is a term used to describe disorders caused by the presence of abnormal hemoglobin production in the blood of a subject. In particular, a hemoglobinopathic disorder or the severity of symptoms of a hemoglobinopathic disorder are amelioratable by re-activation of expression of one or more fetal γ-globin genes in a post-partum mammalian subject. Reference to “post-partum” in this context means a non-fetal mammalian subject. Examples of hemoglobinopathic conditions include β-thalassemia, α-thalassemia, δβ-thalassemia, sickle cell anaemia, HbE, anaemia, Hb caserta, Hb C-Harlem, Hb C and AS, Koln's unstable hemoglobin.

The term “PRMT-5” or “protein methyltransferase-5” means a protein arginine methyltransferase which methylates arginine residues during post-translational modification of proteins. See for example Pollack et al, J. Biol. Chem. 274:31531, 1999; Febbrizio et al, EMBO Rep. 3:641, 2002; and Pal et al, Mol. Cell. Biol. 24:9630, 2004. PRMT-5 regulates transcription of γ-genes by histone methylation and in particular di-methylation of arginine 3 (R3) on histone 4 (H4R3me2s). The complex comprising PRMT-5 is referred to as the PRMT-5-dependent, transcription-regulating complex or the PRMT-5 complex.

Whilst not intending on limiting the present invention to any one theory or mode of action, it is proposed that the PRMT-5-dependent complex comprising Dnmt3a, caseine kinase IIα, Suv4-20n1/2 and α-components of the MBD2/NuRD complex induces phosphorylation of H4S1, tri-methylation of H4K20, K3K9 and H3K27 and CpG methylation.

It is proposed, therefore, to provide antagonists of PRMT-5 activity including enzymatic activity and function and/or its co-factors; of the ability of PRMT-5 to interact with the complex and in particular Nf-E4 (which is a γ-gene promoter binding protein involved in activation and repression of γ-globin genes; Nf-E4 and PRMT-5 co-localize at the γ-gene promoter); of the PRMT-5-containing complex itself; or which inhibit expression of the gene encoding PRMT-5 or which inhibit any other component of the PRMT-5 complex or genes encoding same.

The terms “γ-gene” and “γ-globin gene” and “gene encoder γ-globin” all refer to the group of genes encoding fetal γ-globin. All such terms may be used interchangeably throughout the specification. Since the γ-genes represent a collection of genes, reference herein to “γ-gene” includes one or more than one or a family of γ-genes.

It is proposed in accordance with the present invention that mammalian subjects with abnormal hemoglobin may be treated in such a way so as to reactivate expression of the silenced fetal γ-gene(s). Silencing of expression of the γ-genes occurs after birth.

Hence, one aspect of the present invention contemplates a method for the treatment of a hemoglobinopathy in a mammalian subject, the method comprising administering to the subject an agent which disrupts or down-regulates the activity of a component of a PRMT-5-dependent, transcription complex or a gene encoding PRMT-5 the agent being administered for a time and under conditions sufficient for a repressed fetal γ-globin gene to be expressed.

Another aspect of the present invention provides a method for the treatment of a hemoglobinopathy in a mammalian subject, the method comprising administering to the subject an agent which disrupts or down-regulates the enzymatic or protein binding activity of PRMT-5 or other component in the PRMT-5 complex or expression of a gene encoding PRMT-5 or the other component said agent being administered for a time and under conditions sufficient for a repressed fetal γ-globin gene to be expressed.

The present invention extends to a method for reactivating expression of a silenced γ-globin gene in a cell said method comprising contacting the cell with an agent which disrupts or down-regulates the activity of a compound of a PRMT-5-dependent, transcription complex or a gene encoding PRMT-5.

Reference to “activity” includes enzymatic activity or function and the ability to interact with other components.

Reference to a “PRMT-5-dependent, transcription complex” includes a complex of PRMT-5 (with or without co-factors), Dnmt3a, caseine kinase IIα, Suv4-2oh1/2 and α-components of the MBD2/NuRD complex which complex induces phosphorylation of H4S1, tri-methylation of H4K20, H3K9, and H3K27 and induces CpG methylation. This in turn induces gene silencing of the γ-gene. Antagonists may be to PRMT-5 or any other above-listed components. Particular target components include PRMT-5 or a co-factor thereof and Dnmt3a. However, the present invention extends to any component in the complex or the complex itself.

As used herein, the term “effective amount” means an amount of agent of the present invention effective to yield a desired therapeutic response, for example to induce expression of a silenced γ-gene and/or to prevent or treat or ameliorate the symptoms of a hemoglobinopathic disease.

The specific “effective amount” will of course vary with such factors as the particular condition being treated, the physical condition and clinical history of the subject, the type of mammal being treated, the duration of the treatment, the nature of concurrent therapy (if any), and the specific formulations employed and the structure of the compound.

However, effective amounts of from 0.01 ng/kg subject to 10 g/kg subject may be contemplated. The agent may be a chemical compound, protein or peptide or nucleic acid (i.e. genetic) agent. It may also be a cartilaginous fish-derived immunoglobulin-like molecule such as a shark- or ray-derived immunoglobulin new receptor antigen (IgNAR). See Greenbert et al, NATURE 374:168-173, 1995; Nuttall et al, Mol. Immunol. 38:313-326, 2001; International Patent Publication No. WO 2005/118629. A “genetic agent” also includes a viral construct engineered to enter a cell and release a nucleic acid molecule and/or cause the release or generation of proteinaceous molecules. A “genetic agent” includes RNAi constructs, both DNA-derived or synthetic, as well as antisense constructs.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, of a compound as provided herein is a salt that this not biologically or otherwise undesirable. The carrier may be liquid or solid, and is selected with the planned manner of administration in mind.

The terms “treating” and “treatment” as used herein refer to reduction in severity and/or frequency of symptoms, elimination of symptoms and/or underlying cause, prevention of the occurrence of symptoms and/or their underlying cause and improvement or remediation of damage. Thus, for example, “treating” a patient involves prevention of a particular hemoglobinopathic disorder or adverse physiological event in a susceptible or affected individual as well as treatment of a clinically symptomatic individual by reactivating expression of a fetal γ-globin gene or family or suite of γ-genes.

The agent of the present invention may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease.

The term “subject” as used herein refers to any mammal having a disease or condition which requires treatment with the pharmaceutically-active agent to re-activate fetal γ-globin gene expression. The subject may be a mammal, preferably a primate and most preferably a human.

The present invention, therefore, contemplates methods for treating, hemoglobinopathic diseases related to altered hemoglobin in a subject which treatment comprises re-activating expression of (a) fetal γ-globin gene(s) by targeting PRMT-5 activity or its ability to interact with a fetal γ-globin gene expression repressing complex or function of the complex or levels of PRMT-5 or a component of the PRMT-5 complex or gene encoding same. In some embodiments, modulating the activity or expression of PRMT-5 involves administering an effective amount of an agent that can inhibit PRMT-5 activity or PRMT-5 gene expression. Such agents are described in more detail herein below.

The present invention contemplates methods for identifying a test agent that can modulate PRMT-5 activity (including enzymatic activity) in a test cell comprising contacting the test cell with a test agent and observing wether PRMT-5 is modulated relative to activity in a control cell that was not contacted with the test agent.

Any cell type or test agent available to one skill in the art can be employed. In some embodiments the cell can be an embryonic cell, a cancer cell or an immune cell. In other embodiments, the cell can be a cultured cell that is engineered to express a γ-globin cDNA.

Screening assays for PRMT-5 inhibitors also include a histone H4 arginine 3 (H4R3), the target substitute of PRMT-5 involved in γ-globin gene silencing. For example, a peptide from the N-terminus of histone H4 is synthesized with a biotin tag. This peptide is coupled to streptavidin-coated plate and incubated with recombinant PRMT-5, derived from E. coli.

S-adenosyl-L-methyl-³H-methionine is used as the methyl donor in a mixture of HMTase buffer (25 mM NaCl, 25 mM Tris, pH 8.8). Plates are then washed and individual wells counted for radionucleotide incorporation. The addition of Adox to the incubation mixture provides a positive control for an inhibitory molecule. Non-radioisotopic alternatives for large-scale screening may also be employed. These include the use of a specific antibody to methylated H4R3 (anti-H4R3me2s), with detection via either direct fluorescence, a fluorescent secondary antibody or fluorescence via FRET.

Inhibitory compounds identified in this screen are validated for specificity using other recombinant PRMTs and their specific substrates. The structures of these compounds are also examined with a view to designing molecules with greater specificity, biological activity, bioavaiaobility, etc.

Animal models are conveniently used for testing of “lead” compounds.

The premier model for testing lead compounds is the primate, Paio anubis, the baboon. The effects of potential PRMT-5 antagonists are examined on fetal globin gene expression in this model. Subsequently, lead compounds are also tested.

A mouse model of human fetal hemoglobin production may also be used. The mice are transgenic for a 250-kb yeast artificial chromosome containing the human β-globin locus. although these animals do express the γ-globin genes, the developmental pattern is unlike humans in that the genes are silenced in utero.

The fetal erythroid cell line K562 also provides a facile cellular model for compound validation.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to he quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a preferred approach, the atomic coordinates of three-dimensional structure are used for rational drug design. Modeling can be used to generate modulators (activators and inhibitors) which interact with the linear sequence or a three-dimensional configuration.

A template molecule is generally selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimization or modification can then be carried out to arrive at one or more final mimetics for in vivo or clinical testing.

The present invention contemplates, therefore, methods of screening for agents which modulate PRMT-5 activity or interactactivity with other compounds in the PRMT-5 suppression complex. The PRMT-5-containing complex may itself be inhibited or targeted. PRMT-5 and the complex are also referred to herein as “targets”, “a target” or “target molecule”. The screening procedure includes assaying for the presence of a complex between the drug and the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase.

As indicated above, the present invention extends to IgNAR molecules to PRMT-5 or a component of the PRMT-5-containing complex.

In another embodiment, the immunoglobulin-like molecules comprise the variable domain of an IgNAR referred to as a “V_{NAR}”. The immunoglobulin-like molecules of the present invention enable the selective targeting of the PRMT-5-containing complex and its components.

Reference to a “cartilaginous fish” includes a member of the families of shark and ray. Reference to a “shark” includes a member of order Squatiniformes, Pristiophoriformes, Squaliformes, Carcharinformes, Laminiformes, Orectolobiformes, Heterodontiformes and Hexanchieformes. Whilst not intending to limit the shark to any one genus, immunoglobulins from genus Orectolobus are particularly useful and include the bamboo shark, zebra shark, blind shark, whale shark, nurse shark and Wobbegong. Immunoglobulins from Orectolobus maculates (Wobbegong) are exemplified herein.

The “immunoglobulins” from cartilaginous fish may be referred to herein as “immunoglobulin-like” to emphasize that the cartilaginous fish-derived molecules are structurally different to mammalian or avian-derived immunoglobulins. See Nuttal et al, 2003 supra. For brevity, all cartilaginous fish-derived immunoglobulin-like molecules are referred to herein as “IgNARs”. The variable domain from an IgNAR is referred to as a V_{NAR}™

Reference to “derived” includes vaccination of a fish and collection of blood or immune sera or other body fluid as well as the generation of molecules via recombinant means. By “recombinant means” includes generation of cartilaginous fish-derived nucleic acid libraries and biopanning expression libraries (such as phagemid libraries) for IgNAR proteins which interact with PRMT-5 or a co-factor thereof or a component in the PRMT-5-containing complex.

The present invention also contemplates the use of competitive drug screening assays in which mammalian-, avian- or cartilaginous-derived antibodies capable of specifically binding the target compete with a test compound for binding to the target or fragments thereof. In this manner, the antibodies can be used to detect the presence of any peptide or non-proteinaceous molecule which shares one or more antigenic determinants of the target. The antibodies may also be used to discriminate between various forms of the PRMT-5 complex.

Another embodiment screens computationally small molecule databases for chemical entities or compounds that can bind in whole, or in part, to PRMT-5 or a complex comprising same. This screening method and its utility is well known in the art. For example, such computer modelling techniques were described in a PCT application WO 97/16177.

Once identified by modelling, the agonist/antagonist may then be tested for biological activity. For example, the molecules identified may be introduced via standard screening formats into biological activity assays to determine the inhibitory activity of the compounds, or alternatively, binding assays to determine binding. One particularly preferred assay format is the enzyme-linked immunosorbent assay (ELISA). This and other assay formats are well known in the art and thus are not limitations to the present invention.

It is also possible to isolate a target-specific antibody including an antibody to a particular site or to different forms of PRMT-5-containing complex selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Pursuant to the present invention, such stereochemical complementarity is characteristic of a molecule which matches intra-site surface residues or other binding region identified herein. By “match” is meant that the identified portions interact with the surface residues, for example, via hydrogen bonding or by entropy-reducing van der Waals interactions which promote desolvation of the biologically active compound within the site, in such a way that retention of the biologically active compound within the groove is energetically favoured.

In general, the design of a molecule possessing stereochemical complementarity can be accomplished by means of techniques which optimize, either chemically or geometrically, the “fit” between a molecule and a target. Suitable such techniques are known in the art. (See Sheridan and Venkataraghavan, Acc. Chem. Res. 20:322, 1987; Goodford, J. Med. Chem. 27:557, 1984; Beddell, Chem. Soc. Reviews:279, 1985; Hol, Angew. Chem. 25:767, 1986 and Verlinde, W.G.J. Structure 2:677, 1994, the respective contents of which are hereby incorporated by reference.)

Thus, there are two particular approaches to designing a molecule according to the present invention, which complements the shape of a target binding site. In the first of these, the geometric approach, the number of internal degrees of freedom, and the corresponding local minima in the molecular conformation space, is reduced by considering only the geometric (hard-sphere) interactions of two rigid bodies, where one body (the active site) contains “pockets” or “grooves” or “clefts” which form binding sites for the second body (the complementing molecule, as ligand). The second approach entails an assessment of the interaction of different chemical groups (“probes”) with the active site at sample positions within and around the site, resulting in an array of energy values from which three-dimensional contour surfaces at selected energy levels can be generated.

The geometric approach is illustrated by Kuntz et al, J. Mol. Biol. 161:269-288, 1982, the contents of which are hereby incorporated by reference, whose algorithm for ligand design is implemented in a commercial software package distributed by the Regents of the University of California and further described in a document, provided by the distributor, entitled “Overview of the DOCK Package, Version 1.0,”, the contents of which are hereby incorporated by reference. Pursuant to the Kuntz algorithm, the shape of the cavity represented by the copper-binding site is defined as a series of overlapping spheres of different radii. One or more extant databases of crystallographic data, such as the Cambridge Structural Database System maintained by Cambridge University (University Chemical Laboratory, Lensfield Road, Cambridge CB2 IEW, U.K) and the Protein Data Bank maintained by Brookhaven National Laboratory (Chemistry Dept. Upton, N.Y. 11973, U.S.A.), is then searched for molecules which approximate the shape thus defined.

Molecules identified in this way, on the basis of geometric parameters, can then be modified to satisfy criteria associated with chemical complementarity, such as hydrogen bonding, ionic interactions and van der Waals interactions.

The chemical-probe approach to ligand design is described, for example, by Goodford supra 1984, the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the sites of interest with different chemical probes, e.g., water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3 DB Unity (Tripos Associates, St. Louis, Mo.).

Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from a number of sources including Cambridge Crystallographic Data Centre (Cambridge, U.K.) and Chemical Abstracts Service (Columbus, Ohio).

De novo design programs include Ludi (Biosym Technologies Inc., San Diego, Calif.), Sybyl (Tripos Associates) and Aladdin (Daylight Chemical Information Systems, Irvine, Calif.).

Those skilled in the art will recognize that the design of a mimetic compound may require slight structural alteration or adjustment of a chemical structure designed or identified using the methods of the invention. In addition, the agents may need to be modified to enable penetration into the nucleus of a cell.

This aspect of the present invention may be implemented in hardware or software, or a combination of both. However, the subject invention is preferably implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Hence, the agents of these aspects of the present invention cover antagonists and inhibitors of PRMT-5 enzymatic function and interactability as well as any other component of the PRMT-5 complex.

Any agents that modulate the activity of PRMT-5 or expression of a gene encoding PRMT-5 can be utilized in the subject invention. Such agents can act directly or indirectly on the PRMT-5 gene or on PRMT-5 or PRMT-5-containing complex or on components of PRMT-5-containing complex. Such agents can act at the transcriptional, translational or protein level to modulate the activity including enzymatic activity PRMT-5 or expression of the gene encoding PRMT-5. The term “modulate” or “modulating” means changing, that is increasing or decreasing. Hence, while agents that can decrease PRMT-5 gene expression or PRMT-5 activity can be used in the compositions and method of the invention, agents that also increase PRMT-5 expression or activity are also encompassed within the scope of the invention. The latter agents are more likely to be used in animal models or as research tools.

For the sake of brevity, a gene encoding PRMT-5 is referred to herein as the expression of PRMT-5 or PRMT-5 expression.

In other embodiments, one of skill in the art may choose to decrease PRMT-5 expression, translation or activity. For example, the degradation of PRMT-5 mRNA may be increased upon exposure to small duplexes of synthetic double-stranded RNA through the use of RNA interference (siRNA or RNAi) technology. A process is, therefore, provided for inhibiting expression of a PRMT-5 gene in a cell. The process comprises introduction of RNA with partial or fully double-stranded character into the cell or into the extracellular environment. Inhibition is specific to PRMT-5 RNA because a nucleotide sequence from a portion of the PRMT-5 gene (including its promoter) is chosen to produce inhibitory RNA. This process is effective in producing inhibition of PRMT-5 gene expression.

siRNAs can be designed using the guidelines provided by Ambion (Austin, Tex.). Briefly, the PRMT-5 cDNA sequence is scanned for target sequences that have AA dinucleotides. Sense and anti-sense oligonucleotides can be generated to these targets that contain a G/C content, for example, of about 35 to 55%. These sequences can then be compared to others in the human genome database to minimize homology to other known coding sequences (e.g. by performing a BLAST search using the information available through the NCBI database). siRNAs designed in this manner can be used to modulate PRMT-5 expression.

Mixtures and combinations of such siRNA molecules are also contemplated by the invention. These compositions can be used in the methods of the instant invention, for example, for treating or preventing hemoglobinopathic conditions.

The siRNA selectively hybridizes to RNA in vivo or in vitro. A nucleic acid sequence is considered to be “selectively hybridizable” to a reference nucleic acid sequence if the two sequences specifically hybridize to one another under physiological conditions or under moderate stringency hybridization and wash conditions. In some embodiments, the siRNA is selectively hybridizable to an RNA (e.g. a PRMT-5 RNA) under physiological conditions. Hybridization under physiological conditions can be measured as a practical matter by observing interference with the function of the RNA. Alternatively, hybridization under physiological conditions can be detected in vitro by testing for siRNA hybridization using the temperature (e.g. 37° C.) and salt conditions that exist in vivo.

Moreover, as an initial matter, other in vitro hybridization conditions can be utilized to characterize siRNA interactions. Exemplary in vitro conditions include hybridization conducted as described in the Bio-Rad Labs ZetaProbe manual (Bio-Rad Labs, Hercules, Calif., USA); Sambrook et al, Molecular Cloning: A Laboratory Manual 2^{nd} ed., Cold Spring Harbour Laboratory Press, 1989 or Sambrook et al, Molecular Cloning: A Laboratory Manual, 3^{rd} ed., Cold Spring Harbour Laboratory Press, 2001, expressly incorporated by reference herein.

For example, hybridization can be conducted in 1 mM EDTA, 0.25 M Na₂HPO₄ and 7% w/v SDS at 42° C., followed by washing at 42° C. in 1 mM EDTA, 40 mM NaPO₄, 5% w/v SDS and 1 mM EDTA, 40 mM NaPO₄, 1% w/v SDS. Hybridization can also be conducted in 1 mM EDTA, 0.25 M Na₂HPO₄ and 7% w/v SDS at 60° C., followed by washing in 1 mM EDTA, 40 mM NaPO₄, 5% w/v SDS and 1 mM EDTA, 40 mM NaPO₄, 1% w/v SDS. Washing can also be conducted at other temperatures including temperatures ranging from 37° C. to at 65° C., from 42° C. to at 65° C., from 37° C. to at 60° C., from 50° C. to at 65° C., from 37° C. to 55° C., and other such temperatures.

The siRNA employed in the compositions and methods of the present invention may be synthesized either in vivo or in vitro. In some embodiments, the siRNA molecules are synthesized in vitro using methods, reagents and synthesizer equipment available to one of skill in the art. Endogenous RNA polymerases within a cell may mediate transcription in vivo or cloned RNA polymerase can be used for transcription in vivo or in vitro. For transcription from a transgene or an expression construct in vivo, a regulatory region may be used to transcribe the siRNA strands. Hence, synthetic and DNA-derived siRNA are contemplated by the present invention.

Depending on the particular sequence utilized and the dose of double-stranded siRNA material delivered, the compositions and methods may provide partial or complete loss of function for the target gene (PRMT-5). A reduction or loss of gene expression in at least 99% of targeted cells has been shown for other genes, e.g. U.S. Pat. No. 6,506,559. Lower doses of injected material and longer times after administration of the selected siRNA may result in inhibition in a smaller fraction of cells.

The siRNA may comprise one or more strands of polymerized ribonucleotide; it may include modifications to either the phosphate-sugar backbone or the nucleoside. The double-stranded siRNA structure may be formed by a single self-complementary RNA strand or two complementary RNA strands. siRNA duplex formation may be initiated either inside or outside the cell. The siRNA may be introduced in an amount that allows delivery of at least one copy per cell. Higher doses of double-stranded material may yield more effective inhibition.

Inhibition is sequence-specific in that nucleotide sequences corresponding to the duplex region of the RNA are targeted for genetic inhibition. siRNA containing nucleotide sequences identical to a portion of the target gene is preferred for inhibition. However, siRNA sequences with insertions, deletions, and single point mutations relative to the target sequence may also be effective for inhibition and are encompassed by the present invention. Thus, sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

The siRNA may be directly introduced into the cell, i.e. intracellularly; or introduced extracellularly into a cavity, interstitial space, into the circulation of a subject, introduced orally, or may be introduced by bathing a subject or part thereof in a solution containing siRNA. Methods for oral introduction include direct mixing of siRNA with oral supplements, as well as engineered approaches in which viral constructs are employed. Physical methods of introducing nucleic acids include injection directly into the cell or extracellular injection into the subject of an siRNA solution.

The siRNA may also be delivered in vitro to cultured cells using transfection agents available in the art such as lipfectamine or by employing viral delivery vectors such as those from lentiviruses. Such in vitro delivery can be performed for testing purposes or for therapeutic purposes. For example, cells from a patient can be treated in vitro and then re-administered to the patient.

The advantages of using siRNA include: the ease of introducing double-stranded siRNA into cells, the low concentration of siRNA that can be used, the stability of double-stranded siRNA and the effectiveness of the inhibition.

Anti-sense nucleic acids can also be used to inhibit the expression of a PRMT-5 gene. In general, the function of PRMT-5 RNA is inhibited, for example, by administering to a mammal a nucleic acid that can inhibit the functioning of PRMT-5 RNA. Nucleic acids that can inhibit the function of PRMT-5 RNA can be generated from coding and non-coding regions of the PRMT-5 gene. However, nucleic acids that can inhibit the function of a PRMT-5 RNA are often selected to be complementary to PRMT-5 nucleic acids that are naturally expressed in the mammalian cell to be treated with the methods of the present invention. In some embodiments, the nucleic acids that can inhibit PRMT-5 RNA function are complementary to PRMT-5 sequences found near the 5′ end, 3′ end or internal to the PRMT-5 gene/RNA sequence.

A nucleic acid that can inhibit the functioning of a PRMT-5 RNA need not be 100% complementary to the PRMT-5 RNA. Instead, some variability in the sequence of the nucleic acid that can inhibit the functioning of a PRMT-5 RNA is permitted. For example, a nucleic acid that can inhibit the functioning of a PRTM-5 RNA from a human can be complementary to a nucleic acid encoding either a human or another mammalian PRMT-5 gene product.

Moreover, nucleic acids that can hybridize under moderately or highly stringent hybridization conditions to a nucleic acid comprising the PRMT-5 gene/RNA sequence are sufficiently complementary to inhibit the functioning of a PRMT-5 RNA and can be utilized in the methods of the instant invention.

“Stringent hybridization conditions” and “stringent hybridization wash conditions” in the context of nucleic acid hybridization are somewhat sequence dependent, and may differ depending upon the environmental conditions of the nucleic acid. For example, longer sequences tend to hybridize specifically at higher temperatures. An extensive guide to the hybridization of nucleic acids is found in Tijsssen, Laboratory Techniques in Biochemstry and Molecular Biologly Hybridzation with Nucleic Acid Probes 1(2), Elsevier, N.Y. 1993, Sambrook et al, supra 1989, Sambrook et al, supra 2001.

Generally, highly stringent hybridization and wash conditions are selected to be about 5° C. lower than the thermal melting point (Tₘ) for the specific double-stranded sequence at a defined ionic strength and pH. The Tₘ is the temperature (under defined ionic strength and pH) at which 50% of a complementary target sequence hybridizes to a perfectly matched probe. For example, under “high stringent conditions” or “highly stringent hybridization conditions” a nucleic acid will hybridize to its complement to a detectably greater degree than to other sequences (e.g. at least 2-fold over background). By controlling the stringency of the hybridization and/or washing conditions nucleic acids that are 100% complementary can be hybridized.

For DNA-DNA hybrids, the Tₙ, can be approximated from the equation of Meinkoth and Wahl, Anal. Biochem. 138:267-284, 1984:

Tₘ=81.5° C.+16.6(log M)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs.

Very stringent conditions are selected to be equal to the Tₘ for a particular probe. Alternatively, stringency conditions can be adjusted to allow some mismatching in sequences so that lower degrees of similarity can hybridize. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destablizing agents such as formamide.

Exemplary low stringency conditions include hybridization with a buffer solution of 30 to 35% v/v formamide, 1 M NaCl, 1% w/v SDS (sodium dodecyl sulfate) at 37° C. and a wash in 1× to 2×SSC (20×SSC=3.0 M NaCl and 0.3 M trisodum citrate) at 50 to 55° C. Exemplary moderate stringency conditions include hybridization in 40 to 45% formamide, 1.0 M NaCl, 1% w/v SDS at 37° C., and a wash in 0.5× to 1×SSC at 55 to 60° C. Exemplary high stringency conditions include hybridization in 50% v/v formamide, 1 M NaCl, 1% w/v SDS at 37° C. and a wash in 0.1×SSC at 60 to 65° C.

The degree of complementarity or sequence identity of hybrids obtained during hybridization is typically a function of post-hybridization washes, the critical factors being the ionic strength and temperature of the final wash solution. The type and length of hybridizing nucleic acids also affects whether hybridization will occur and whether any hybrids formed will be stable under a given set of hybridization and wash conditions.

An example of stringent hybridization conditions for hybridization of complementary nucleic acids that have more than 100 complementary nucleic acids that have more than 100 complementary residues on a filter in a Southern or Northern blot is 50% v/v formamide with 1 mg of heparin at 42° C. with the hybridization being carried out overnight. An example of highly stringent conditions is 0.15 minutes. Often, a high stringency wash is preceded by a low stringency wash to remove background probe signal. An example of medium stringency for a duplex of, e.g. more than 100 nucleotides, is 1×SSC at 45° C. for 15 minutes. An example of low stringency wash for a duplex of, e.g. more than 100 nucleotides, is 4-6×SSC at 40° C. for 15 minutes. For short probes (e.g. about 10 to 50 nucleotides), stringent conditions typically involve salt concentrations of less than about 1.0 M Na ion, typically about 0.01 to 1.0 M Na ion concentration (other salts) at pH 7.0 to 8.3, and the temperature is typically at least about 30° C.

Stringent conditions can also be achieved with the addition of destablizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical. This occurs, e.g. when a copy of a nucleic acid is created using the maximum codon degeneracy permitted by the genetic code.

The present invention provides, therefore, a method for identifying a test agent that can modulate PRMT-5 expression in a cell comprising contacting the cell with a test agent and observing whether expression of a PRMT-5 encoding nucleic acid is modulated relative to expression of a nucleic acid in a cell that was not contacted with the test agent.

The present invention extends to antibodies and other immunological agents directed to or preferably specific for PRMT-5 or which distinguish between PRMT-5 present or absent complex or a component thereof or a particular level of complex or a fragment thereof. The antibodies may be monoclonal or polyclonal or may comprise Fab fragments or synthetic forms. Such antibodies are not likely to be useful therapeutic agents but are useful in screening assays for PRMT-5 or a inhibitor thereof.

Techniques for the assays contemplated herein are known in the art and include, for example, sandwich assays and ELISA.

It is within the scope of this invention to include any second antibodies (monoclonal, polyclonal or fragments of antibodies or synthetic antibodies) directed to the first mentioned antibodies referred to above. Both the first and second antibodies may be used in detection assays or a first antibody may be used with a commercially available anti-immunoglobulin antibody.

Both polyclonal and monoclonal antibodies are obtainable by immunization with PRMT-5 or a complex containing same. PRMT-5 or components or complexes thereof or antigenic fragments thereof are utilizable in immunoassays. The PRMT-5 may need to be conjugated to a carrier molecule. The methods of obtaining both types of sera are well known in the art. Polyclonal sera are less preferred but are relatively easily prepared by injection of a suitable laboratory animal with an effective amount of subject polypeptide, or antigenic parts thereof, collecting serum from the animal and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

A biological sample includes a cell extract.

Immunoassays may be conducted in a number of ways such as by Western blotting and ELISA procedures. A wide range of immunoassay techniques are available as can be seen by reference to U.S. Pat. Nos. 4,016,043, 4,424,279 and 4,018,653.

The PRMT-5 antagonizing agents of the present invention, including their salts, as well as the PRMT-5 siRNA, ribozymes, sense and anti-sense nucleic acids are administered to modulate PRMT-5 expression or activity, or to achieve a reduction in at least one symptom associated with a condition, indication, infection or disease associated with hemoglobinopathy. Other agents can be included such as agents which describe a PRTM-5-containing complex.

In some embodiments the therapeutic agent of the invention are administered in a “therapeutically effective amount”. Such a therapeutically effective amount is used herein to identify an amount sufficient to obtain the desired physiological effect, e.g. treatment of a condition, disorder, disease and the like or reduction in symptoms of the condition, disorder disease and the like.

Administration of the therapeutic agents in accordance with the present invention may be a single dose, in multiple doses, in a continuous or intermittent manner, depending, for example, upon the recipient's physiological condition, wether the purpose of the administration is therapeutic or prophylactic, and other factors known to skilled practitioners. The administration of the therapeutic agents and compositions of the invention may be essentially continuous over a preselected period of time or may be in a series of spaced doses. Both local and systemic administration is contemplated.

Thus, one or more suitable unit dosage forms comprising the therapeutic agents of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agents may also be formulated for sustained release (for example, using microencapsulation, see WO 94/07529 and U.S. Pat. No. 5,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-sold carriers, finely divided solid carriers or combinations thereof, and then, if necessary, introducing or shaping the product into the desired delivery system.

When the therapeutic agents of the invention are prepared for oral administration, they are generally combined with a pharmaceutically acceptable carrier, diluent or excipient to form a pharmaceutical formulation, or unit dosage form. For oral administration, the therapeutic agents may be present as a powder, a granular formulation, a solution, a suspension, an ingestion of the active ingredients from a chewing gum. The therapeutic agents may also be presented as a bolus, electuary or paste. Orally administered therapeutic agents of the invention can also be formulated for sustained release, e.g. the therapeutic agents can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic agents of the invention can be prepared by procedures known in the art using well-known and readily available ingredients. For example, the therapeutic agents can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methlcellulose and other cellulose derivatives, alginates, gelatin, and poltyvinyl-pyrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cety71 alcohol and glycerol monosterate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum Arabic, or alternatively polyethylene glycols, bentones and montmorillonites and the like.

For example, tablets or caplets containing the therapeutic agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magensiu7m sterate, microcrystalline cellulose, starch, talc, titanium dioxide, benzoic acid, citric acid, corn starch, mineral oil, polypropylene glycol, sodium phosphate, zine stearate, and the like. Hard or soft gelatin capsules containing at least one therapeutic agent of the invention can contain inactive ingredients such as gelatin, microcrystalline cellulose, sodium lauryl sulfate, starch, talc, and titanium dioxide, and the like, as well as liquid vehicles such as polyethylene glycols (EPGs) and vegetable oil. Moreover, enteric-coated caplets or tablets containing one or more therapeutic agents of the invention are designed to resist distintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g. by injection, for example, bolus injection or continuous infusion) and may be presented in unit does form in ampoules, pre-filled syringes, small volume infusion containers or in multi-dose containers. As noted above, preservatives can be added to help maintain the shelf life of the dosage form.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isorpropyl myristate, animal, mineral and vegetable oils and polysiloxanes.

It is possible to add, if necessary, an adjuvant chosen from antioxidants, surfactants, other preservatives, fil-forming, keratolytic or comedolytic agents, perfumes, flavorings and colorings. Antioxidants such as t-butylhyroquinone, butylated hydroxyanisole, butylated hydroxytoluene and -tocopherol and its derivatives can be added.

The present invention is directed to the use of PRMT-5 or a complex comprising same or a component thereof or a gene encoding PRMT-5 or other component in the manufacture of a medicament for the treatment of a hemoglobinopathic condition in a mammal such as a human subject.

The nucleotide and corresponding amino acid sequence of PRMT-5 are shown in SEQ ID NOs:12 and 13, respectively.

The present invention is further described by the following non-limiting Examples. In these Examples, the materials and methods outline below may be employed.

### Cell Culture and Immunofluorescence

K562 cells were grown in RPMI medium 1640 supplemented with 10% fetal bovine serum at 37° C. and in 5% v/v CO₂ supplemented with 50 U of penicillin/ml and 50 μg of streptomycin/ml. CD34⁺ cells isolated from fresh CB were cultured in IMDM supplemented with 15% v/v fetal calf serum (FCS), SCF (100 ng/ml), EPO (5 U/ml), IGF-1 (40 ng/ml) and Dexamethasone (1 μM) to induce erythroid differentiation. CD34⁺ cells isolated from fresh adult BM were cultured in IMDM supplemented with 15% v/v FCS, SCF (100 ng/ml), IL-3 (10 ng/ml), and Flt-3 ligand (500 ng/ml) for seven days, followed by EPO (5 U/ml) alone for five days to induce erythroid differentiation. The relative levels of γ-globin as determined by Q-RT-PCR normalized to HPRT in CB versus BM cultures was 14:1. For immunofluorescence, CB and BM erythroid progenitors were mounted on polylysine slides and permeabilized with 0.1% v/v Triton X-100. Slides were incubated with mouse monoclonal anti-PRMT-5 antibody overnight at 4° C., washed and incubated with Texas Red conjugated horse anti-mouse secondary antibody (Viector Laboratories, Burlingame, Calif., USA) for 1 hour at room temperature. Slides were washed and counterstained with DAPI for 3 minutes prior to maging with a Zeiss Axioplan microscope (Zeiss, Jena, Germany). Experiments utilizing human tissues were approved by the Melbourne Health Human Research Ethics Committee.

### Mass Spectrometry

FLAG immunoprecipitates from K562 cells expressing NF-E4-FLAG were resolved on a 4-20% w/v gradient SDS-PAGE gel and stained with SimplyBlue Safestain (Invitrogen, Carlsbad, Calif., USA). Protein bands of interest were excised from preparative 1D gels and extensively washed in deionized water. Excised gel bands were digested with trypsin. Digests were dried to ˜10 μL by centrifugal lyophilization (Savant model AES1010, Thermo, Waltham, Mass., USA) ready for electrospray-Ion Trap (ESI-IT) tandem mass spectrometry (MS/MS) (LCQ-Deca, Finnigan, San Jose, Calif., USA). Protein digests (˜10 μL of 1% v/v formic acid) were transferred into 100 μL glass autosampler vials and peptides were fractionated by capillary reversed-phase (RPO-HPLC (Agilent Model 1100 capillary HPLC) using a butyl-silica 150×0.15 mm I.D> RP-capillary column (ProteCol™-C4, 3 μm, 300 Å SGE, Australia) developed with a linear 60 minute gradient from 0-100% B, where Solvent A was 0.1% v/v aqueous formic acid and Solvent B was 0.1% v/v aqueous formic acid/60% v/v ACN with a flow rate of 0.8 μL/min. The capillary HPLC was coupled on-line to the ESI-IT mass spectrometer for automated MS/MS analysis of individually isolated peptide ions (Moritz et al, Electrophoresis 17:907, 1996). Uninterpreted CID spectra were filtered excluding spectra with less than 10 peaks using the LCQ-DTA program as part of Bioworks 3.1 srl (Finnigan). The parameters used to create the peak lists are as follows: minimum mass 400; maximum mass 5000; grouping tolerance 1.5; intermediate scans 1; minimum group count 1; LCQ-DTA auto charge state calculation; 10 peaks minimum per spectrum; peptide charge states 1+, 2+ and 3+; ±2 Da peptide mass tolerance; ±0.5 Da MS/MS fragment mass tolerance. Parent ion masses were determined based on the isotope cluster spacing in the zoom scan spectrum and individual spectra files (.dta file extension) were generated. These files were then automatically searched using Mascot™ version 2.1 (Matrix Science, U.K.) against the latest LudwigNR database (Moritz et al, Anal. Chem. 76:4811, 2004). Searches were conducted with the carboxymethylation of cysteine as a fixed modification (+58 Da), variable oxidation if methionine (+16 Da) and the allowance of up to three missed tryptic cleavages. Peptide identifies were chosen to be correct with mascot scores of at least 40 and above but were also manually validated according to the fragmentation principles as previously published (Kapp et al, Anal. Chem. 75:6251, 2003).

### Immunoprecipitation and Immunoblotting

Cells were lyzed in ice-cold lysis buffer (150 mM NaCl, 50 mM Tris-HCl pH8.0, 1 mM EDTA, 1% v/v NP-40, 10 mM sodium butyrate) containing a protease inhibitor cocktail (Roche Diagnostics, Mannheim, Germany) and cleared by centrifugation. Immunoprecipitations were carried out by adding the stated antisera plus protein G-sepharose beads, followed by incubation at 4° C. The immunoprecipitates were washed extensively, subjected to SDS-PAGE, and transferred to PVDF membranes. The membranes were incubated with various specific antibodies, then washed extensively prior to incubation with peroxidase-conjugated anti-rabbit or anti-mouse immunoglobulin G. After further extensive washes, the blots were visualized by using ECL reagents (Amersham Biosciences, Amersham, U.K.). All immunoprecipitations were performed in duplicate. Antibodies utilized in the immunoprecipitations—FLAG (Sigma-Aldrich, St. Louis, Mo., U.S.A), HA (Roche), PRMT-5, CKIIα, Suv4-20h1/2, MBD2, MeCP2 (Abcam), Mi2, mSin3A, Dnmt3a, Dnmt3b, MBD3, Brg-1, tubulin, GATA-1 (Santa Cruz, Santa Cruz, Calif., U.S.A), HDAC1 (Upstate, Charlottesville, Va., U.S.A). Many of these were also utilized in ChIP assays.

### Recombinant Protein Expression and GST Pull-Down Assay

GST-fusion proteins were produced in BL21 E. coli as previously described (Zhou et al, Mol. Cell. Biol. 20:7662, 2000). [³⁵S] labeled PRMT-5 synthesized using the T7 TNT kit (Promega, Madison, Wis., U.S.A) and Trans ³⁵S label (ICN, Irvine, Calif., U.S.A) were incubated with GST fusion proteins pre-bound to glutathione beads at 4° C. overnight. The beads were washed extensively and subjected to SDS-PAGE. The gels were dried and analyzed by autoradiography.

### ChIP Analysis

ChIP assays were performed as previously described (Zhao et al, Blood 107:2138, 2006). Isolated DNA fragments were purified with a QIAquick spin kit (QIAGEN, Hilden, Germany) and 2 μl from a 40 μl DNA extraction was amplified quantitatively by real time PCR with the γ-globin gene promoter specific primers or MyoD primers as a negative control. Primers for the globin, MyoD and GATA-1 promoter sequences are available upon request. Antibodies specific for various post-translational modifications of the histone tails utilized were H4R3me2s, H4S1ph, H4K20me3, H3K9me3, H3K27me3 (Abcam), H4 Pan, H4K5ac, H4K5ac, H4K8ac, H4K12ac, H4K16ac (upstate), RNA PolII (Santa Cruz).

### In Vitro Methyltransferase Assays

Beads from immunoprecipitation assays from K562 cells transfected with PRMT-5-f or PRMT-5Δ-f and HA-NF-E4 were processed as previously described (Rea et al, Nature 406:593, 2000), with slight modifications. Briefly, 10 μg of purified histone H2A, H2B, H3 and H4 (Roche) as substrates and 2 μCi of S-adenosyl-L-methyl-³H-methoionine (³H-SAM; Amersham) as the methyl donor, were incubated in a mixture of 20 μl of HMTase buffer (25 mM NaCl, 25 mM Tris, pH 8.8) for 2 hours at 30° C. Proteins were resolved on a 14% SDS-PAGE gel, stained with Coomassie blue, and then dried and subjected to autoradiography.

### Bisulfite Sequence Analysis

Bisulfite sequence analysis was performed as previously described (Lavelle et al, supra 2006). Primers to amplify the bisulfite treated γ-promoter:

| | |
|---|---|
| Sense Forward | |
| 5′-TATGGGTTGGTTAGTTTTGTTTTG-3′ | (SEQ ID NO: 1) |
| Sense Reverse | |
| 5′-CACATTCACCTTACCCCACAA-3′ | (SEQ ID NO: 2) |
| Antisense Forward | |
| 5′-GTTTGGATTAGGAGTTTATTGATA-3′ | (SEQ ID NO: 3) |
| Antisense Reverse | |
| 5′-TTCCCCACACTATCTCAAT-3′ | (SEQ ID NO: 4) |

PCR was performed with HiFi Taq polymerase (Roche) as follows: 30 cycles, 94° C. for 20 s, 55° C. for 20 s and 68° C. for 35 s. PCR products were cloned into pCRII (Invitrogen) followed by nucleotide sequencing using the Big-Dye Termination method (ABI, Columbia, Md., U.S.A).

### RNA Interference and Retroviral Infections

The siRNA target sequence for PRMT-5 was inserted into the pSUPER.retro.neo+gfp retroviral vector according the manufacturer's recommendations (oligoEngine, Seattle, Wash., U.S.A).

The oligo sequences were:

PRMT-5 siRNA—GGACCTGAGAGATGATATA (SEQ ID NO:5) and GAGGATTGCAGTGGCTCTT (SEQ ID NO:6), scrambled control ACGTCTACTATCGACCCC (SEQ ID NO:7).

Retrovirus production by 293T cells and infection of K562 cells were performed as described (Zhao et al, supra 2006).

Transduced cells were selected fro GFP expression by FACS.

### RT-PCR and Real Time PCR

Total RNA was isolated from cells with Trizol reagent (Invitrogen). cDNA was generated by using the reverse transcription system (Promega).

Quantitative real-teim RT-PCR (Q-RT-PCR) primers:

| | |
|---|---|
| HPRT: sense | |
| 5′-ATGGACAGGACTGAACGTCT-3′ | (SEQ ID NO: 8) |
| HPRT antisense | |
| 5′-CTTGCGACCTTGACCATCTT-3′ | (SEQ ID NO: 9) |
| γ-globin: sense | |
| 5′-AGCTTTGGCAACCTGTCCTCT-3′ | (SEQ ID NO: 10) |
| γ-globin: antisense | |
| 5′-GGCCACTCCAGTCACCATCTT-3′ | (SEQ ID NO: 11) |

Q-RT-PCR was done in a Rotorgene 2000 (Corbett Research, Sydney, Australia), in a final volume of 20 Reaction mixtures contained 1× times reaction buffer, 2.5 mM MgCl2, 0.5 mM deoxynucleotides (Roche), 0.1 μM gene-specific primers, 1 U Taq polymerase (Fisher Biotech), a 1:10,000 dilution of SYBR Green I (Molecular Probes) and 2 μl of sample or standard.

### Example 1

### Role of Symmetric Di-Methylation of Histone H4 Arginine 3 in Epigenetic Control of Mammalian Gene Silencing

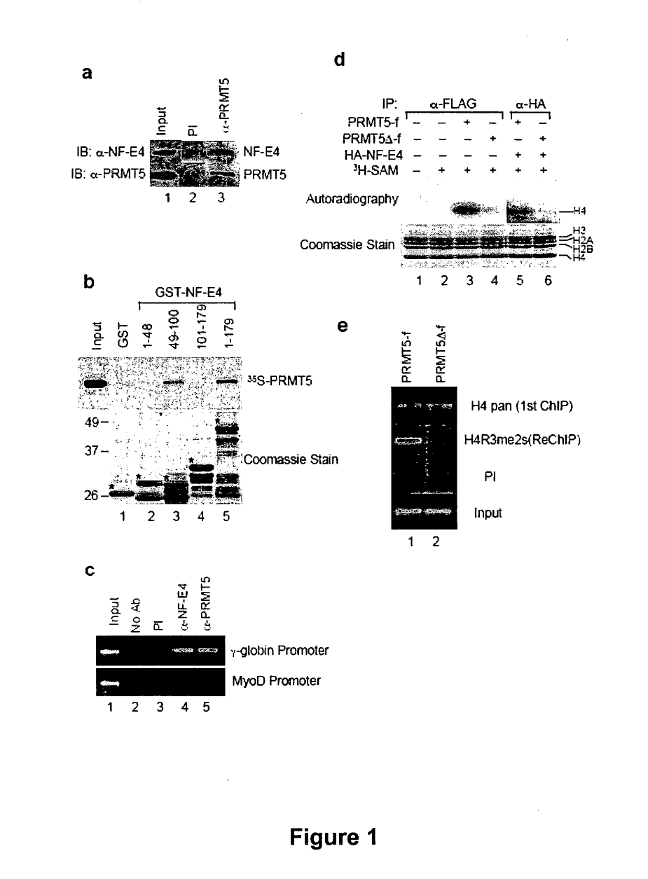

The β-globin locus has served as a paradigm for analyzing the role of epigenetic modifications in the regulation of tissue and developmentally-specific gene expression (Litt et al, supra 2001; Johnson et al, supra 2001). In both humans and primates, the fetal (γ)-globin genes are progressively silenced after birth, displaying methylation of a cluster of CpG dinucleotides in the proximal promoters and 5′ untranslated regions in adult bone marrow (van der Ploeg and Flavell, supra 1980). Reversal of this methylation is associated with fetal globin gene reactivation (Lavelle et al, supra 2006). In co-immunoprecipitation experiments designed to identify proteins complexed with NF-E4, a proximal γ-promoter-binding protein implicated in both activation and repression of the γ-globin genes Zhou et al, supra 2000; Zhao et al, supra 2006), multiple peptides of the protein methyltransferase, PRMT-5 (Pollack et al, supra 1999) were identified by mass spectrometry. The interaction between these two proteins was confirmed by co-immunoprecipitation of epitope tagged proteins expressed in the human fetal erythroid cell line, K562 and of the endogenous proteins from the same cell line (FIG. 1A). The interaction between PRMT-5 and NF-E4 was direct, as demonstrated by GST-chromatography (FIG. 1B) and involved the region of NF-E4 unique to the full-length isoform. This isoform has been shown to bind to the proximal γ-globin promoters in the setting of γ-gene repression (Zhao et al, supra 2006). NF-E4 and PRMT-5 were co-localized at the γ-promoters by chromatin immunoprecipitation (ChIP) in K562 cells using antisera to the endogenous proteins (FIG. 1C).

PRMT-5 is an arginine methyltransferase that has been implicated in gene silencing through the establishment of repressive histone marks including symmetrical di-methylation of arginine 3 on histone H4 (H4R3me2s) and histone H2A (H2AR3me2s) [Pollack et al, supra 1999; Fabbrizio et al, supra 2002] and arginine 8 on histone H3 (H3R8me2s) [Pal et al, supra 2004]. To identify the histone substrates of PRMT-5 in K562 cells, lines were derived expressing FLAG-tagged PRMT-5 (PRMT-5-f), or a mutant containing a five amino acid deletion in the S-adenosyl-L-methionine binding motif that lacks methyltransferase activity (PRMT-5Δ-f Pollack et al, supra 1999). Immunoprecipitates generated with anti-FLAG antisera were subjected to a standard radioactive histone methyltransferase activity assay (Rea et al, supra 2000), which demonstrated radio labeling of histone H4 with wild-type, but not mutant PRMT-5 (FIG. 1D, left panel). Methylation of the other known substrates of PRMT-5 (histones H2A and H3) was not detected in this context. To determine whether this methyltransferase activity was associated with NF-E4, K562 cell lines were generated expressing hemagglutinin epitope (HA)-tagged NF-E4, and either PRMT-5-f or PRMT-5Δ-f. Comparable levels of expression in these lines were confirmed by western blot with anti-FLAG and anti-HA antisera. Incubation of purified histones with immunoprecipitated derived with anti-HA antisera also specifically labeled histone H4 in the context of wild-type but not mutant PRMT-5 expression (FIG. 1D, right panel). To determine whether K562 cells expressing PRMT-5-f, but not PRMT-5Δ-f displayed the specific repressive epigenetic mark H4R3me2s at the γ-promoters, CUP analysis was performed (FIG. 1E). As the antibody utilized for this assay (ab5823, Abcam, Cambridge, U.K.) recognizes symmetric methylation of R3 on both histone H4 and H2A (Ancelin et al, Nat. Cell. Biol. 8:623, 2006), ChIP/ReChIP was performed with a pan H4 antibody followed by ab5823 to specifically quantitated H4R3me2s at the γ-promoters. Although the levels of H4 were identical in the two cell lines, a substantial increase in H4R3me2s in cells expressing wild-type PRMT-5 compared to the mutant was observed.

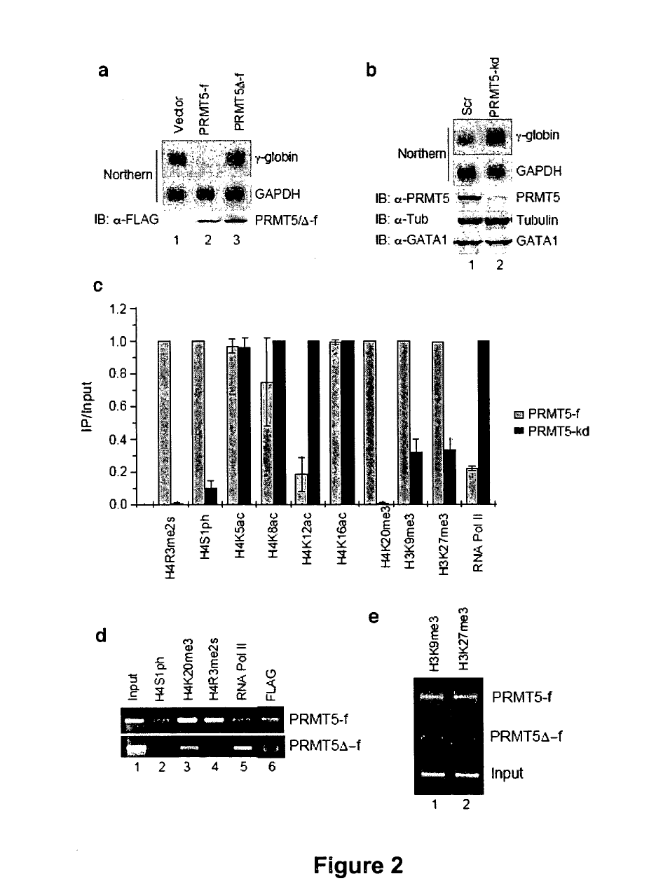

Although K562 cells are used as a model of feal erythropoiesis as they express the γ- but not the β-globin genes, only a relatively small percentage of cells (10%) express globin chains when grown in the absence of the chemical inducer, hemin. To determine the effects of perturbed expression of PRMT-5 on γ-gene expression, Northern analyses on PRMT-5-f and PRMT-5Δ-f expressing K562 cells was performed (FIG. 2A, top panels). Expression of both proteins was confirmed by immunoblotting with anti-FLAG antisera (FIG. 2A, bottom panel). Enforced expression of PRMT-5-f induced almost complete silencing of γ-gene expression. In contrast, expression of PRMT-5Δ-f led to a four-fold induction of γ-gene expression compared to the vector control. To confirm this finding, PRMT-5 expression in K562 cells was knock down using two different stably expressed short interfering RNAs (siRNAs) (PRMT-5-kd). Cells transfected with an expression vector containing a scrambled sequence served as the control (scr). Western blotting confirmed that PRMT-5 protein levels were reduced by more than 90% in the PRMT-5-kd cells compared with the scrambled control, but not effect was observed on the control proteins, tubulin or GATA-1 (FIG. 2B, lower panels). The knock down of PRMT-5 led to a four-fold induction of γ-gene expression compared to the scrambled siRNA vector (FIG. 2B, upper panels).

To determine whether additional histone modifications were induced in response to increased PRMT-5 expression, chip analyses was performed on the PRMT-5-f and PRMT-5-kd K562 lines using a range of specific antibodies (FIG. 2C). Consistent with the γ-gene expression data, the H4R3me2s repressive mark was readily detected at the γ-promoters in the PRMT-5-f cells, but was completely absent at the promoters in the PRMT-5-kd cells. Two other histone H4 modifications that have been linked to gene silencing, phosphorylation of H4S1 (H4S1ph) [Utley et al, Mol. Cell. Biol. 25:8179, 2005] were also differentially localized to the γ-promoters in the two lines, with high levels observed in the PRMT-5-f lines, and absence of the marks in the knock down lines. Tri-methylation of H3K9 (H3K9me3), which has previously been shown to be required for the H4K20me3 repressive mark (Schotta et al, Genes Dev. 18:1251, 2004), and tri-methylation of H3K27 (H3K27me3), which has been linked to DNA methylation (Fuks, supra 2005), were also increased in the PRMT-5-f lines. These changes were reflected in the localization of RNA polymerase II (RNA pollII) to the promoter, which was markedly reduced in the PRMT-5-f cells compared to the knock down cells. Assessment of histone H4 acetylation in the different cell lines revealed a reduction in H4K12ac in the PRMT-5-f cells, but no alterations at lysines 5, 8 or 16. No changes in any of the histone marks were observed at either the MyoD or GATA-1 promoters.

Whether the presence of these repressive histone markers were dependent on the methyltransferase activity of PRMT-5 was examined by performing ChIP analyses on the K562 cells expressing PRMT-5Δ-f compared with PRMT-5-f. Binding of the mutant PRMT-5 to the γ-promoters in these cells, was demonstrated which was accompanied by loss of H4R3me2s. Concomitantly, complete loss of H4S1ph was observed, and a marked reduction of H4K20me3 at the γ-promoters, coincident with reduced levels of RNA PolII (FIG. 2D). The levels of H3K9me3 and H3K27me3 were also markedly reduced, suggesting that these marks were established as a consequence of H4R3me2s induced by PRMT-5 (FIG. 2E). These findings indicated that the methyltransferase activity of PRMT-5 and not just its physical occupation of the promoters, was integral for the subsequent generation of repressive histone marks.

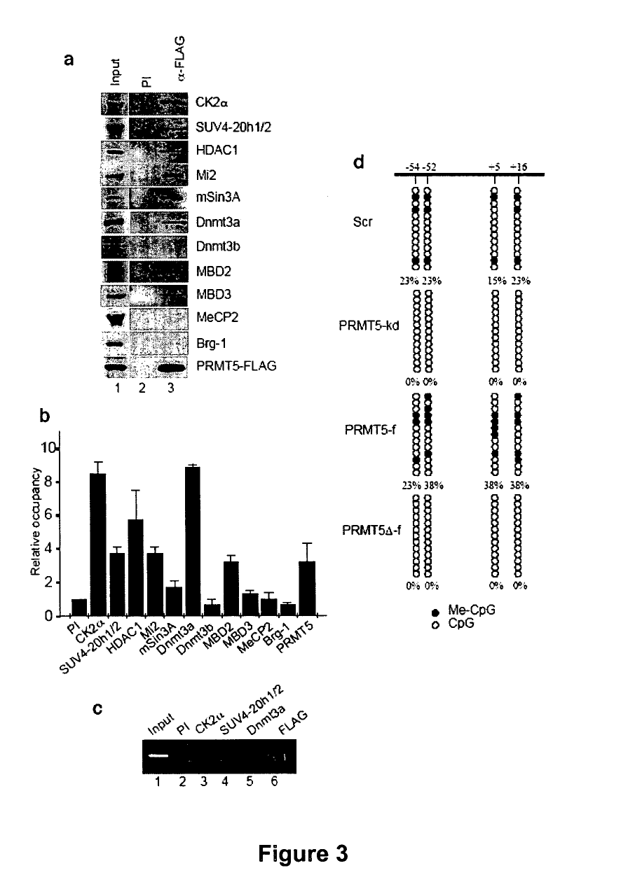

PRMT-5 has been linked to transcriptional repression through the formation of two multi-protein complexes, one containing mSin3A, HDAC2 and SWI/SNF components Brg1 and Brm (Pal et al, supra 2004; Pal et al, Mol. Cell. Biol. 23:7475, 2003) and the other containing MBD2 and components of the NuRD complex (Le Guezennec et al, Mol. Cell. Biol. 26:843, 2006). To determine whether these factors associated with PRMT-5 in K562 cells, immunoprecipitations were performed with extract from the PRMT-5-f cells using the anti-FLAG antisera, and blotted the precipitates with antibodies to a range of candidate protein partners (FIG. 3A). Associates between PRMT-5 and the MBD2/NuRD complex proteins Mi2, mSin3A, MBD2 and HDAC1 were demonstrated. MBD3 was also identified as a PRMT-5 interacting protein this setting. In contrast, Brg-1 was not associated with PRMT-5. The DNA methyltransferase Dnmt3a was demonstrated to be associated with PRMT-5. Casein kinase IIα (CKIIα) and Suv4-20h1/2, the enzymes linked to the repressive markers H4S1ph and H4K20me3s identified in ChIP analysis were also found to co-immunoprecipitate with PRMT-5 (Utley et al, supra 2005; Schotta et al, supra 2004). The localization of these complex components on the γ-promoters was confirmed by ChIP in K562 cells expressing PRMT-5-f (FIG. 3B).

In view of the reduction in H4S1ph and H4K20me3s in cells expressing PRMT-5Δ-f, the possibility of whether assembly of the repressor complex on the γ-promoters was also dependent on the methyltransferase activity of PRMT-5 was examined. ChIP assays were performed on PRMT-5Δ-f expressing cells using antisera to selected complex components (FIG. 3C). The results indicated that the methyltransferase activity of PRMT-5 was essential for assembly of the repressor complex, and the subsequent repressive histone marks that emanated from this.

The presence of Dnmt3a in the PRMT-5-dependent complex raised the possibility that repression of γ-gene expression by this complex may also involve DNA methylation. To determine this, the methylation status of the promoters in the PRMT-5-f, PRMT-5-kd, and PRMT-5Δ-f stable cell lines were examined using bisulfite DNA sequencing, with the line stably transfected with the scrambled siRNA construct serving as the control (FIG. 3D). Consistent with the observation that globin chain expression is not detectable in a large proportion of uninduced K562 cells, methylation of the four CpG dinucleotides immediately flanking the transcriptional start site in 15-23% of clones derived from the scrambled construct transfection was observed. This frequency was increased in three of the four sites in the PRMT-5-f lines, with 38% of the clones showing methylation. In contrast, methylation of all the CpG dinucleotides was abolished in clones derived from the PRMT-5-kd cells, suggesting that PRMT-5 is essential for the epigenetic modification of DNA in this setting. Complete loss of methylation was also observed in the PRMT-5Δ-f cells in keeping with data showing loss of both complex assembly and additional repressive modifications in the absence of PRMT-5 enzymatic activity.

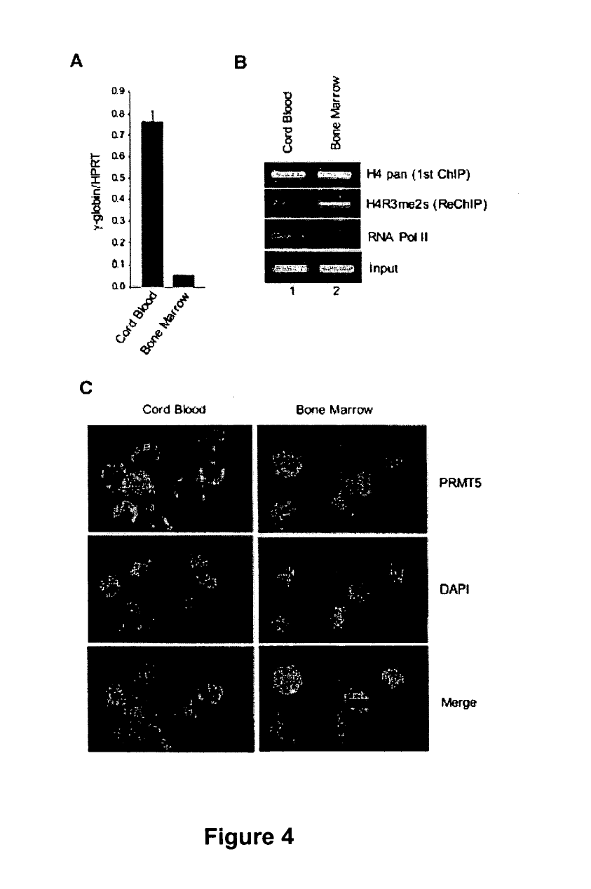

To determine whether the repressive histone mark induced by PRMT-5 was evident at the human γ-globin promoters in a developmentally specific pattern, primary erythroid progenitors were isolated from cord blood (CB) and adult bone marrow (BM). Expression of the γ-globin genes was markedly higher in CB compared to BM by quantitative RT-PCR (FIG. 4A). ChIP/ReChIP analysis using the pan H4 antisera followed by ab5823 demonstrated an increase in H4R3me2s at the γ-promoters in adult BM erthroid progeinotrs compared with progenitors derived from CB (FIG. 4B). This was accompanied by a reduction in RNA polII localized to the γ-promoters. PRMT-5 has been shown previously to translocate from the nucleus to the cytoplasm in mouse germ cells at the time of extensive epigenetic reprogramming of mouse germ cells (Ancelin et al, supra 2006). The cellular localization of PRMT-5 was examined by immunofluorescence in the CB and BM erythroid progenitors and demonstrated that the protein was predominantly nuclear in the BM, whereas it was primarily localized in the cytoplasm in the CB progenitors (FIG. 4C). These findings indicate a mechanism by which PRMT-5 may play a specific developmental role in regulating the human β-globin locus.

This example establish that arginine methylation of histones can be closely linked to DNA methylation, in addition to a range of other repressive epigenetic marks. The assembly for these modifications is dependent on the initial symmetric methylation of H4R3 mediated by PRMT-5, which induces the formation of a multi-protein complex containing PRMT-5 and Dnmt3a. The presence of MBD2, MBD3, HDAC1 and other repressors in this complex may serve to reinforce the silencing. The identification of CKIIα and Suv4-20h1/2 in the repressor complex suggests that PRMT-5 induces coordinated epigenetic events, with the establishment of the repressive markers H4S1ph, H4K20me3s, H3K9me3, H3K27me3 and 5meCpG at the promoters. This recruitment is dependent on the methyltransferase activity of the protein, as PRMT-5Δ-f, although retaining the ability to localize to the γ-promoters, is unable to mediate the assembly of the repressor complex.

The studies indicate that H4R3me2s is a key early step in establishment of the repressive domain. Taken together, these findings indicate that these PRMT-5, acting through histone H4R3, plays contrasting roles in the developmental regulation of the β-globin locus.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ancelin et al, Nat. Cell. Biol. 8:623, 2006
Beddell, Chem. Soc. Reviews:279, 1985
Bird, Genes Dev 16:6, 2002
Bulger et al, Mol Cell Biol 23:5234, 2003
Febbrizio et al, EMBO Rep. 3:641, 2002
Fuks, Curr. Opin. Genet. Dev. 15:490, 2005
Goodford, J. Med. Chem. 27:557, 1984
Greenbert et al, NATURE 374:168-173, 1995
Hol, Angew. Chem. 25:767, 1986
Kuntz et al, J. Mol. Biol. 161:269-288, 1982
Jackson et al, Nature 416:556, 2002
Johnson et al< Mol. Cell. 8:465, 2001
Kapp et al, Anal. Chem. 75:6251, 2003
Lavelle et al, Exp. Hematol 34:339, 2006
Le Guezennec et al, Mol. Cell. Biol. 26:843, 2006
Lehnertz et al, Curr. Biol. 13:1192, 2003
Litt et al, Science 293:2453, 2001
Pal et al, Mol. Cell. Biol. 24:9630, 2004
Pal et al, Mol. Cell. Biol. 23:7475, 2003
Pollack et al, J. Biol. Chem. 274:31531, 1999
Meinkoth and Wahl, Anal. Biochem. 138:267-284, 1984
Moritz et al, Electrophoresis 17:907, 1996
Moritz et al, Anal. Chem. 76:4811, 2004
Nuttall et al, Mol. Immunol 38:313-326, 2001
Rea et al, Nature 406:593, 2000
Sambrook et al, Molecular Cloning: A Laboratory Manual 2^{nd} ed., Cold Spring Harbour Laboratory Press, 1989
Sambrook et al, Molecular Cloning: A Laboratory Manual, 3^{rd} ed., Cold Spring Harbour Laboratory Press, 2001
Schneider et al, Nat. Cell. Biol. 6:73, 2004
Schotta et al, Genes Dev. 18:1251, 2004
Sheridan and Venkataraghavan, Acc. Chem. Res. 20:322, 1987
Tamaru et al, Nat. Genet. 34:75, 2003
Tijsssen, Laboratory Techniques in Biochemstry and Molecular Biologly Hybridzation with Nucleic Acid Probes 1(2), Elsevier, N.Y. 1993
U.S. Pat. No. 6,506,559
U.S. Pat. No. 5,962,091
Utley et al, Mol. Cell. Biol. 25:8179, 2005
van der Ploeg and Flavell, Cell 19:947, 1980
Verlinde, W.G.J. Structure 2:677, 1994
WO 94/07529
WO 2005/118629
Zhao et al, Blood 107:2138, 2006
Zhou et al, Mol. Cell. Biol. 20:7662, 2000

1. A method for the treatment of a hemoglobinopathy in a mammalian subject, said method comprising administering to said subject an antagonist of protein methyltransferase-5 (PRMT-5) or an antagonist of PRMT-5 gene expression said antagonist being administered for a time and under conditions sufficient for a suppressed fetal γ-globin gene to be expressed.

2. A method of claim 1 wherein a silenced γ-globin gene in a cell is expressed,

3. The method of claim 1, wherein the antagonist inhibits formation of a PRMT5-dependent repressor complex.

4. The method of claim 1, wherein the antagonist de-methylates a γ-globin gene.

5. The method of claim 1, wherein the antagonist binds to or inactivates PRMT-5 and inhibits its enzymatic activity.

6. The method of claim 5, wherein the antagonist is an antisense construct, a ribozyme or an siRNA.

7. The method of claim 5, wherein the antagonist is an antibody, a PRMT-5 peptide mimetic, a peptide, a peptide aptamer or a small molecule.

8. The method of claim 7, wherein the antibody comprises a single chain.

9. The method of claim 7, wherein the antibody is a monoclonal antibody.

10. The method of claim 7 wherein the antibody is a cartilaginous fish-derived antibody.

11. The method of claim 10 wherein the antibody is an immunoglobulin new receptor antigen (IgNAR).

12. The method of claim 1, wherein the hemoglobinopathy is selected from the group consisting of β-thalassemia, α-thalassemia, δβ-thalassemia, sickle cell anaemia, anaemia, Hb caserta, Hb C-Harlem, Hb C and AS, Koln's unstable hemoglobin.
